(19) United States
(12) Patent Application Publication
(10) Pub. No.: US 2009/0187209 A1
(43) Pub. Date: Jul. 23, 2009

(54) MEANS AND METHOD OF REMOVAL OF EMBOLIC MATERIAL FROM A BLOOD VESSEL

(76) Inventor: Jose Cohen, Jerusalem (IL)

Correspondence Address: Fleit Gibbons Gutman Bongini & Bianco PL, 21355 EAST DIXIE HIGHWAY, SUITE 115, MIAMI, FL, 33180, US

(21) Appl. No.: 12/096,211

(22) PCT Filed: Dec. 5, 2006
(86) PCT No.: PCT/IL06/01397

§ 371 Date: Sep. 2, 2008

Related U.S. Application Data
(60) Provisional application No. 60/742,903, filed on Dec. 7, 2005.

(51) Int. Cl. A61B 17/22

(52) U.S. Cl. 606/200

(57) ABSTRACT

The present invention discloses a mechanical embolectomy device for the removal of intravascular embolic material, especially blood clots. The device comprises of a microcatheter of dimensions suitable for introduction into a blood vessel; a microguide wire; and a retriever coil attached to the distal end of said microcatheter; said retriever coil is positioned to dislodge and remove said embolic material from said blood vessel. A method for removing of intravascular embolic materials is also disclosed.

### FIELD OF THE INVENTION

The present invention generally relates to a means and method of removal of embolic material from a blood vessel. More particularly, but not exclusively, it relates to the removal of blood clots leading to ischemic stroke.

### BACKGROUND OF THE INVENTION

Approximately 750,000 strokes occur in the United States annually, of which 85% are ischemic. Ischemic stroke may be caused by occlusion of a large intracranial arteries (>2 mm), and large-vessel occlusions carry a particularly high mortality estimated between 53% and 92%. Most ischemic strokes are attributable to cerebrovascular propagation of embolic material which consists of platelets, red cells, atheromatous debris, or a mixture of these elements. These emboli originate either in the heart, aortic arch or supraaortic trunks. Whether permanent neurological injury will occur after an arterial occlusion depends on the duration and degree of reduction in local blood flow and the intrinsic vulnerability of the affected cells to the ischemic injury. Based on the widely accepted concepts of infarction and ischemic penumbra, emergent treatments of cerebral ischemia have focused upon the preservation and recovery of the ischemic penumbra by limiting the amount of neuronal tissue damage caused by the ischemic process.

Reopening large cerebral vessels would be expected to reduce neurological morbidity and mortality if performed before ischemic brain damage is maximal. Neurological improvement has been demonstrated for patients treated with thrombolytic drugs administered either intravenously or intra-arterial, however many patients are ineligible for thrombolytic therapy and in many other patients thrombolytic therapy is ineffective.

A mechanical embolectomy device could be an alternative to stroke therapy, especially in patients in whom thrombolytic therapy in contraindicated or unsuccessful. Several endovascular mechanical techniques for clot removal or lysis have been developed, and some especially designed devices are currently undergoing clinical trials. These include the use of lasers, angioplasty, ultrasonography, microsnares, and devices that can physically grasp and remove a thrombus from the cerebral circulation. However, only a few of them have shown procedural safety, significant revascularization rates and clinical efficacy.

Thus there remains a long felt need for the present invention relating to a means and method of removal of embolic material from a blood vessel.

### SUMMARY OF THE INVENTION

It is therefore one objective of the present invention to disclose a mechanical embolectomy device for the removal of intravascular embolic material, particularly but not exclusively blood clots in the brain leading to ischemic stroke. This device comprises a microguide wire, a microcatheter of dimensions suitable for introduction into a blood vessel and a retriever coil attached to the distal end of said microcatheter such that the retriever coil is positioned in order to dislodge and remove the embolic material from the blood vessel.

Another objective of the present invention is to teach a method of removing of intravascular embolic material, particularly blood clots, by introducing the retriever coil into the region of the blood clot, dislodging the embolic material from the blood clot and removing said embolic material from the blood vessel such that blood flows unimpeded through the blood vessel.

A further objective of the present invention is to teach a method of removing of intravascular embolic material where the said microguide is introduced into a blood vessel as far as the blood clot, the microcatheter and retriever coil are passed along the length of the microguide wire until the tip of the retriever coil is placed in the central portion of the blood clot. Using the retriever coil the embolic material from the clot is dislodged and a mild inward aspiration through the microcatheter is applied such that the embolic material is dislodged from the clot and removed from the blood vessel through the catheter.

### BRIEF DESCRIPTION OF THE FIGURES

In order to understand the invention and to see how it may be implemented in practice, few preferred embodiments will now be described, by way of non-limiting example only, with reference to the accompanying drawing, in which;

FIG. 1 schematically represents the mechanical embolectomy device and,

FIG. 2 schematically represents mechanical embolectomy device during the action of removing embolic material from the blood vessel.

### DETAILED DESCRIPTION OF THE EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method of removal of embolic material from a blood vessel.

The term ‘plurality’ applies hereinafter to any integer greater than or equal to one.

The term ‘about’ applies hereinafter to any value in the range from 20% below to 20% above the stated value.

The term ‘embolectomy device’ applies hereinafter to any device designed to remove embolic material from the body.

The term ‘embolic material’ applies hereinafter to any material carried by blood stream such as platelets, red cells, atheromatous debris, or a mixture of these elements particularly where such material builds up leading to the formation of blood clots.

The term ‘microcatheter’ applies hereinafter to a flexible tube adapted to be introduced into the body.

The term ‘aspiration’ applies hereinafter to the flow of air or any other fluid particularly through a tube.

The term ‘microguide wire’ applies hereinafter to a wire of small diameter adapted to the passed through the body along a defined path.

The term ‘attraumatic’ applies hereinafter to any procedure which causes no damage to surrounding tissue.

The term ‘blood clot’ applies hereinafter to any obstruction within a blood vessel which acts to impede the flow of blood through the blood vessel.

It is according to one embodiment of the present invention to disclose a mechanical embolectomy device for the removal of intravascular embolic material, particularly blood clots, comprising a microguide wire, a microcatheter of dimensions suitable for introduction into a blood vessel and a retriever coil attached to the distal end of said microcatheter such that the retriever coil is positioned in order to dislodge and remove the embolic material from the blood vessel.

It is according to another embodiment of the present invention to disclose a mechanical embolectomy device additionally comprising an aspiration means by which the dislodged embolic material is removed through the microcatheter.

It is according to another embodiment of the present invention to disclose a mechanical embolectomy device wherein the shape of said microguide wire is adapted to the particular route to be taken by the microcatheter.

It is according to another embodiment of the present invention to disclose a mechanical embolectomy device wherein an attraumatic passage through said microcatheter and said retriever coil spring is provided through which the microguide wire passes.

It is according to another embodiment of the present invention to disclose a mechanical embolectomy device wherein said microcatheter is tapered for example from proximal diameter of about 3 F to a distal tip of about diameter 2.4 F.

It is according to another embodiment of the present invention to disclose a mechanical embolectomy device wherein the internal diameter of said microcatheter is about 0.017-inch.

It is according to another embodiment of the present invention to disclose a mechanical embolectomy device wherein the diameter of the microguide wire is about 0.014-inch.

It is according to another embodiment of the present invention to disclose a mechanical embolectomy device wherein the retriever coil is constructed from a strong and chemically inert metallic material such as platinum.

It is according to another embodiment of the present invention to disclose a mechanical embolectomy device additionally comprising a guiding balloon catheter which is used to temporarily occlude the proximal region of the blood vessel during treatment.

It is according to another embodiment of the present invention to disclose a mechanical embolectomy device additionally comprising a mechanical means by which the retriever coil is moved laterally across the blood clot.

It is according to another embodiment of the present invention to disclose a mechanical embolectomy device additionally comprising a means of infusing fibrinolytics through the microcatheter during the procedure.

It is according to another embodiment of the present invention to teach a method of removing of intravascular embolic material, particularly blood clots, by introducing a retriever coil into the region or the blood clot, dislodging the embolic material from the blood clot and removing said embolic material from the blood vessel such that blood flows unimpeded through the blood vessel.

It is according to another embodiment of the present invention to teach a method of removing of intravascular embolic material additionally comprising providing a microguide wire, providing a microcatheter of dimensions suitable for introduction into a blood vessel and providing a retriever coil attached to the distal end of said microcatheter. Said microguide is introduced into a blood vessel as far as the blood clot, the microcatheter and retriever coil are passed along the length of the microguide wire until the tip of the retriever coil is placed in the central portion of the blood clot. Using the retriever coil the embolic material from the clot is dislodged and a mild inward aspiration through the microcatheter is applied such that the embolic material is dislodged from the clot and removed from the blood vessel through the catheter.

It is according to another embodiment of the present invention to teach a method of removing of intravascular embolic material additionally comprising introducing a guiding balloon catheter into the blood vessel and occluding the blood vessel at least partially for the duration of the procedure.

It is according to another embodiment of the present invention to teach a method of removing of intravascular embolic material additionally disrupting the clot through lateral motion of the retrieval coil across the embolic material constituting the clot.

It is according to another embodiment of the present invention to teach a method of removing of intravascular embolic material additionally infusing fibrinolytics into the clot through the microcatheter.

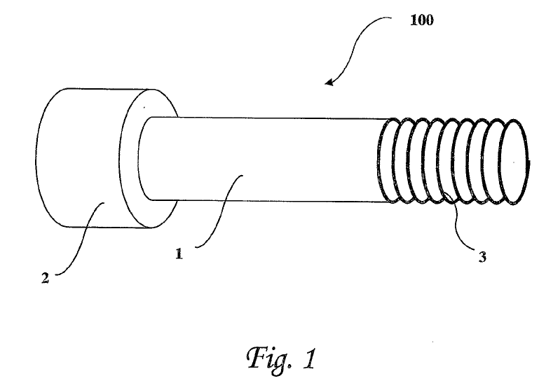

Reference is now made to FIG. 1 schematically representing the mechanical embolectomy device, 100, comprising a microcatheter, 1, of dimensions suitable for introduction into a blood vessel, a microguide wire, 2, and a retriever coil, 3, attached to the distal end of the microcatheter

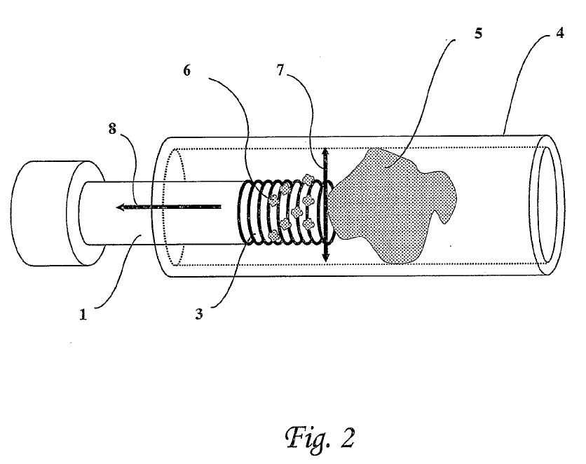

Reference is now made to FIG. 2 schematically representing the mechanical embolectomy device, 100, during the action of removing embolic material, 5, from a blood vessel, 4. Lateral movement, as indicated by the double headed arrow, 7, of the retriever coil, 3, through the embolic material, 5, causes pieces of embolic material to be caught by the retriever coil, 3, and are removed from the blood vessel by withdrawing the microcatheter, 1, in the direction of the arrow, 8.

1. A mechanical embolectomy device (100) for the removal of intravascular embolic material (5), especially blood clots, comprising;
a. a microcatheter of dimensions suitable for introduction into a blood vessel (4);
b. a microguide wire (2); and,
c. a retriever coil (3) attached to the distal end of said microcatheter; said retriever coil is positioned to dislodge and remove said embolic material from said blood vessel.

2. The mechanical embolectomy device, according to claim 1, additionally comprising of an aspiration means by which said dislodged embolic material is removed through said microcatheter.

3. The mechanical embolectomy device, according to claim 1, wherein the shape of said microguide wire is adapted to the particular route to be taken by said microcatheter.

4. The mechanical embolectomy device, according to claim 1, wherein an attraumatic passage through said microcatheter and said retriever coil spring is provided through which said microguide wire passes.

5. The mechanical embolectomy device, according to claim 1, wherein said microcatheter is tapered, especially wherein proximal diameter is about 3 F and distal tip is about diameter 2.4 F.

6. The mechanical embolectomy device, according to claim 1, wherein the internal diameter of said microcatheter is about 0.017 inch.

7. The mechanical embolectomy device, according to claim 1, wherein the diameter of the microguide wire is about 0.014 inch.

8. The mechanical embolectomy device, according to claim 1, wherein the retriever coil is constructed from a strong and chemically inert metallic material such as platinum.

9. The mechanical embolectomy device, according to claim 1, additionally comprising a guiding balloon catheter which is used to temporarily occlude the proximal region of the blood vessel during treatment.

10. The mechanical embolectomy device, according to claim 1, additionally comprising a mechanical means by which the retriever coil is moved laterally across the blood clot.

11. The mechanical embolectomy device, according to claim 1, additionally comprising a means of infusing fibrinolytics through the microcatheter during the procedure.

12. A method of removing of intravascular embolic material, particularly blood clots by;
a. introducing a retriever coil into the region of the blood clot;
b. dislodging the embolic material from the blood clot; and
c. removing said embolic material from the blood vessel, whereas blood flows unimpeded through the blood vessel.

13. The method of removing of intravascular embolic material, according to claim 12, additionally comprising;
a. providing a microguide wire;
b. providing a microcatheter of dimensions suitable for introduction into a blood vessel;
c. providing a retriever coil attached to the distal end of said microcatheter;
d. introducing said microguide wire into a blood vessel as far as the blood clot;
e. passing the microcatheter and retriever coil along the length of the microguide wire until the tip of the retriever coil is placed in the central portion of the blood clot;
f. using the retriever coil to dislodge the embolic material from the clot; and
g. providing a mild inward aspiration through the microcatheter;
such that the embolic material is dislodged from the clot and removed from the blood vessel through the catheter.

14. The method of removing of intravascular embolic material, according to claim 13, additionally comprising;
a. introducing a guiding balloon catheter into the blood vessel; and
b. occluding the blood vessel at least partially for the duration of the procedure.

15. The method of removing of intravascular embolic material, according to claim 13, additionally disrupting the clot through lateral motion of the retrieval coil across the embolic material constituting the clot.

16. The method of removing of intravascular embolic material, according to claim 13, additionally infusing fibrinolytics into the clot through the microcatheter.
